(19) United States
(12) Patent Application Publication
(10) Pub. No.: US 2002/0048573 A1
(43) Pub. Date: Apr. 25, 2002

(54) Lytic enzymes useful for treating fungal infections

(76) Inventors: John C. Klock, Nicasio, CA (US); Chittra Mishra, Southborough, MA (US); Christopher M. Starr, Sonoma, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 09/932,558

(22) Filed: Aug. 17, 2001

Related U.S. Application Data
(63) Continuation-in-part of application No. 09/078,208, filed on May 13, 1998, now abandoned.

(51) Int. Cl. A61K 38/43; A01N 25/00

(52) U.S. Cl. 424/94.1; 424/405

(57) ABSTRACT

The present invention features methods of treating fungal infections in mammals including humans by administering one or more lytic enzymes and compositions comprising the same. The present invention further features a new method for isolating and purifying lytic enzymes to a degree of purity acceptable for treating fungal infections, including invasive Aspergillosis.

This application is a continuation-in-part of U.S. patent application Ser. No. 09/078,208, filed on May 13, 1998.

### FIELD OF THE INVENTION

The present invention is in the field of medicine, carbohydrate chemistry and biochemistry. The present invention further features methods of treating fungal infections in animals and humans by administering lytic enzymes from Trichoderma spp and compositions comprising the same. Furthermore, the present invention features a new method for isolating and purifying such lytic enzymes from Trichoderma spp.

### BACKGROUND OF THE INVENTION

Carbohydrates play many important roles in the functioning of living organisms. In addition to their metabolic roles, carbohydrates are structural components of the cell walls and membranes of plants, animals and microbes. For example, yeast cell walls consist of an outer protein layer of mannoproteins and an inner layer mainly of carbohydrates. Fungal cell walls commonly contain polymers of D-glucose called glucans and polymers of nitrogen containing N-acetyl-D-glucosamine called chitin (Bartnicki-Garcia, Ann Review Microbiol. 22:87-108 (1968)). Glucans may be chemically linked in various ways, most commonly through anomeric (α or β) C1 carbons to the C2, C3, C4 or C6 carbons of a neighboring glucose moiety. This variation in linkage may be expressed as a “β1-3 glucan” or a “β1-6 glucan.” Chitins, however, are only linked in a β1-4 fashion.

The glucans and chitins of fungal cell wall are primarily involved as structural components, maintaining rigidity and conferring protection. The cell wall proteins, β1,6-glucan, and β1,3-glucan are thought to occur in a 1:1:1 ratio and form an elementary building block (cell wall proteins→β1,6-glucan→β1,3-glucan). In addition, a small amount of cell wall proteins is linked to chitin through β1,6-glucan. The reducing end of β1,6-glucan is linked to a non-reducing terminal glucose of β1,3-glucan. In the yeast cell wall, for example, the cell wall proteins are linked by a β1,6-glucan chain of about 140 glucose residues to a β1,3-glucan chain of approximately 1500 sugar residues. After cytokinesis, a chitin chain may become attached to a non-reducing end of the β1,3-glucan chain, thereby further strengthening the cell wall. They may also be degraded and used as nutritional sources after exhaustion of external nutrients. (Kapteyn, et al., Biochim. Biophys. Acta 1426: 373-383 (1999)).

β1,6-Glucans are relatively small molecules and represent only about five percent of the cell wall. β1,6-glucans may play a key role by cross-linking the GPI cell wall proteins to β1,3-glucan, and to a lesser extent to chitin. With respect to the elementary building block, cell wall proteins→β1,6-glucan→β1,3-glucan, when levels of β1,3-glucan are significantly reduced, a greater percentage of cell wall proteins become resistant to β1,3-gluanase extraction as a result of increased formation of the chitin→β1,6-glucan linkage. β1,6-glycosylated cell wall proteins have been observed in several other fungi such as the yeast Hansenula polymorpha, the medically important fungi Aspergillus fumigatus, Candida albicans, and Wangiella (Exophiala) dermatitidis, the plant pathogen Fusarium oxysporum, and in the food spoilage fungi Aspergillus niger, Paecilomyces variotti, and Penicillium roquefortii. The β1,6-glucosylated cell wall proteins are widespread in the largest class of fungi, Ascomycetes. (Kapteyn, et al., Biochim. Biophys. Acta 1426: 373-383 (1999)).

While the majority of the fungi carry out essential activities in nature, some are pathogenic to plants, animals and humans. Fungi cause diseases in animals and humans through several mechanisms. First, some fungi elicit immune responses that can result in allergic reactions. For example, asthma and other hypersensitivity reactions are caused by exposure to specific fungi antigens in the environment. A second fungal disease-causing mechanism involves toxins generated by fungi. For example, afatoxin produced by Aspergillus flavus is highly toxic and induces tumors in some animals. The third mechanism of fungal related disease is through infection. The growth of a fungus on or in the body can cause symptoms that range in severity from relatively innocuous, superficial diseases to serious, life-threatening diseases. In 1996 there were over a million serious fungal infections in the United States. The problem of serious invasive fungal infections has increased with advances in transplantation technology and the growing numbers of patients with immunosuppressive viral infections. Aspergillus infections are common in up to 5% of immunosuppressed patients with mortality rates approaching 90%.

The health implications of indoor fungal contaminants have become a widespread concern in recent years. Relatively well defined diseases associated with fungal contamination of buildings are considered “building-related diseases” (BRI). The “sick building syndrome” (SBS) is commonly associated with environmental fungi. Mycotoxin-producing fungi are not uncommon in residential buildings, and include Aspergillus fumigatus, Stachybotrys atra, Cryptococcus neoformans, Histoplasma, Penicillium spp., Fusarium spp. (Fungal Contamination in Public Buildings: A Guide to Recognition and Management, Federal-Provincial Committee on Environmental Health and Occupational Health, Environmental Health Directorate, Health Canada, Tunney's Pasture, Ottawa, Ontario K1A 0L2, June 1995). In a non-epidemic setting, a significant relationship was found between environmental fungi contamination in hematology wards and the incidence of invasive nosocomial aspergillosis (Alberti, et al., J. Hosp. Infect. 48(3):198-206 (2001)). An increase in invasive aspergillosis was detected among organ transplant recipients, and epidemiologic investigation revealed cases to be both nosocomial and community-acquired (Patterson, et al., Transpl. Infect. Dis. 2(1):22-28 (2000)).

Conventional anti-fungal agents include topical antiseptic chemicals used for non-invasive infections and polyene and azole antibiotics, which inhibit fungal cell wall sterol biosynthesis. Polyenes produced by streptomyces species bind to ergosterol (equivalent of cholesterol in higher eukaryotic cell membranes), which disrupts the normal membrane function and eventually causes membrane permeability and cell death. Azole is a group of antibiotics that selectively inhibit ergosterol biosynthesis. The treatment with azoles results in the inability of fungi to produce a normal membrane, leading to membrane damage and alteration of critical membrane activities. Polyenes and azoles have satisfactory effects on common species of fungi such as Candida, Histoplasma and Coccidiodes, but they exhibit little activity against the invasive Aspergillus which are a common cause of death in the immunosuppressed population. In addition, treatment of fungal infection based on polyenes and azoles is often toxic and has many undesirable side effects. The use of the existing anti-fungal agents has also resulted in the emergency of populations of resistant fungi and the emergency of new pathogenic fungi strains.

A new class of antifungals, the echinocandins, are inhibitors of cell wall synthesis. These new antifungal agents specifically inhibit (1,3)-β-D-glucan synthase, an enzyme complex that forms glucan polymers in the fungal cell wall. (Denning, J. Antimicrobial Chemother. 40:611-614 (1997)). Cancidas (caspofungin acetate) was the first drug of the new echionocandin class of antifungal agents approved by the FDA for treatment of patients unresponsive or intolerable to standard therapies for invasive aspergillosis. Cancidas was not studied as an initial therapy for invasive Aspergillosis and this use was not recommended. Drug-related adverse effects reported in patients treated with Cancidas include fever, phlebitis/thrombophlebitis and/or infused vein complications, headache, nausea, vomiting, rash, skin flushing and mild liver function test elevations, and one case of anaphylaxis (FDA Talk Paper, Jan. 29, 2001, http://www.fda.gov/opacom/hpnews.html).

Thus, it is advantageous to provide novel antibiotics with different mechanisms of action from those in the prior art. These novel antibiotics may be used to broaden the scope of anti-fungal treatment and complement the activities of known anti-fungal agents. They may also be used to treat fungal infections in humans and animals, which are resistant to conventional drugs. The use of lytic enzymes to degrade fugal cell walls as the basis for anti-fugal treatment in animals and humans is the focus of the instant invention.

Many fungi secrete lytic enzymes into their environment as a means to generate food sources from polysaccharides or to gain competitive advantage in their microenvironment by inhibiting the growth of other fungi or parasitizing their neighbors (Haran, et al., Microbiology 142:2321-2331 (1996); Archer et al., Crit. Rev. Biotechnol. 17(4):273-306 (1997)). The inhibition of fungal growth is attributed to lytic enzymes that degrade fugal cell walls and eventually lead to fungal cell lysis. Fungal lytic enzymes include glucanase, chitinases, proteases, lipase, and other hydrolytic enzymes (glucanase and chitinase are glucan-degrading and chitin-degrading enzymes respectively). These lytic enzymes can be further divided into subcategories according to their modes of degradation reaction and type of linkage(s) they degrade, such as, endo- or exo- enzymes and (1,3)-β- or (1,4)-β-enzymes. Lytic enzymes function in an endo fashion cleave the polymeric linkage at random sites along the polysaccharide chain. Those that function in an exo- fashion cleave subsequent polymeric unit from the end. Glucanase and chitinase such as endo and exo (1,3)-β-glucanases, endo and exo (1,4)-β-glucanases, endo and exo (1,6)-β-glucanases, endochitinases, exochitinases, chitobiohydrolases, endochitosanases, exochitosanases, 1,4-β-poly-N-acetyl-D-glucosaminidase, and endo and exo 1,4-β-poly-D-glucosaminidase have been detected in a wide range of fungi species.

Considerable research effort has focused on the studies of lytic enzymes produced by Trichoderms, which are common fungi found in almost any soil. Trichoderms are strongly antagonistic to other fungi, due in part to their secretion of lytic enzymes, such as glucanases, chitinases and proteases, to degrade cell walls of other fungi and in turn utilize their nutrients. As described above, 1,3-β-glucan is one of the main structural components of the fungal cell wall, and 1,3-β-glucanases are secreted by a number of Trichoderma species (Kitamoto, et al., Agric. Biol. Chem. 51:3385-3385 (1987); Dubourdieu, et al., Carbohydr. Res. 144:277-287(1985); Lorito, et al., Phytopathology 84:398-405 (1994); Del Rey, et al., J. Gen. Microbiol. 110:83-89 (1979)).

Many 1,3-β-glucanases have been extensively characterized and studied, and their encoding genes identified and cloned. The involvement of 1,3-β-glucanases in biological control and plant defense mechanisms against fungi has also been well documented (Haran, et al., Microbiology 142:2321-2331 (1996)). 1,6-β-glucanases have been shown to lyse yeast and fungal cell walls. Relatively little information is reported with respect to their purification, characterization, and anti-fungal activities (Haran, et al., Microbiology 142:2321-2331 (1996)). Trichoderma harzianum was shown to produce at least two extracellular 1,6-β glucanase. De la Cruz, et al. were the first to purify one of the two 1,6-β glucanases to homogeneity and to study their hydrolytic activity against fungal cell walls (De la Cruz, et al., J. Bacteriol. 177:6937-6945 (1995)). Several chitinases are secreted by Trichoderma harzianum, and many chitinases have been identified and purified to homogeneity or near homogeneity (Haran, et al., Microbiology 142:2321-2331 (1996)), and their encoding genes cloned and overexpressed (De La Cruz, et al., Eur. J. Biochem. 206:859-867 (1992)).

A variety of glucanases, chitinases, proteases and other hydrolytic enzymes produced by Trichoderma species have been implicated in the biological control of plant fungal pathogens. U.S. Pat. No. 4,062,941 discloses a method for treating fungal infections in animals by administering a fungal lytic enzyme extracted from Coprimus or Lycoperdon, with or without an antimycotic agent, such as amphotericin B, nystatin, or griseofulvin. This and all other U.S. patents cited herein are hereby specifically incorporated herein by reference in their entirety. Pope and Davies (Postgraduate Medical J. 55:674-676 (1979)) teach a method of treating systemic fungal infections in mice by administering fungal lytic enzymes obtained from Coprinus comatus, Physarum polycephalum, and Lycoperdon pyriforme alone or in conjunction with conventional antimycotic drugs such as amphotericin B. Chalkley, et al. (Sabouraudia: J. Med. Vet. Mycol. 23:147-164 (1985)) discloses in vitro antifungal activity of mycolases comprising chitinase, β1,3-glucanase, and exo-glycosidases from Physarum polycephalum against Candida pseudotropicalis and Candida albicans, but only slight enhancement of amphotericin B treatment of mice systemically infected with Candida albicans. In two human subjects infected with pulmonary coccidioidomycosis and one patient with Aspergillus pulmonary and mediastinal infection, mycolases administered with amphotericin B was not effective in eliminating the fungal infections. International Patent Application No. WO 94/13784 discloses an antifungal composition comprising a fungal cell wall degrading enzyme, such as endochitinases, chitin 1,4-β-chitobiosidases, glucan 1,3-β-glucosidases and cellulases, together with a non-enzymatic fungicide, and a method of inhibiting the replication, germination, or growth of a chitin- and 1,3-β-glucan-containing fungus, such as Botrytis cinerea. International Patent Application No. WO 95/31534 and U.S. Pat. Nos. 5,770,406 and 6,022,723 disclose a DNA construct encoding β1,6-endo-glucanase activity, a method of producing said enzyme, a preparation of said enzyme, and a method of using said enzyme in degradation or modification of β-glucan-containing materials. However, no studies have shown the therapeutically effective use of the lytic enzymes isolated from Trichoderms, such as β1,6-glucanase or chitobiosidase, for the in vivo treatment of fungal infection, including invasive Aspergillosis. The treatment based on these lytic enzymes disclosed herein offers a new approach to fighting fungal infections, especially against the more invasive and resistant fungal infections, such as Aspergillosis. Because humans and animals are not known to have glucan or chitin structures like those of lower animals and microbes, glucanases, chitinases and proteases should not display significant toxicity or undesirable biologic effects in humans or animals.

### SUMMARY OF THE INVENTION

In general, the present invention features a method of treating a fungal infection in mammals including humans by extracting lytic enzymes from a fungus, purifying said fungal lytic enzymes to a degree of purity acceptable for therapeutic use in human subjects, formulating one or more of said purified fungal lytic enzymes in a pharmaceutical composition together with a pharmaceutically acceptable carrier; and administering said pharmaceutical compositions to a subject in need thereof an amount therapeutically effective to treat said fungal infection. The present invention further features a new method for isolating lytic enzymes useful for treating fungal infections, preferably these lytic enzymes are isolated preferably these lytic enzymes are purified from Trichoderms. Preferably these lytic enzymes are purified.

In one embodiment, the present invention features methods for treating mammals including humans suffering from fungal or mycoparasitic infections by administering a pharmaceutically effective amount of one or more lytic enzymes such as those obtained from a Trichoderm. Lytic enzymes produced by Trichoderms useful for treating fungal and mycoparasitic infections include glucanases, chitinases, chitosanases and proteases. They are administered in vivo to organisms in order to treat, eliminate or prevent infection by organisms possessing a cell wall such as fungi. Exemplary fungal species that may be treated by the compounds of the present invention include, for example, Aspergillus infections.

In a second embodiment, the present invention features pharmaceutical compositions containing lytic enzyme(s) useful for treating fungal infections. The lytic enzymes of the present invention may be administered alone or in pharmaceutically acceptable compositions to treat infections caused by organisms sensitive to their activities, such as fungi possessing a cell wall. In preferred embodiments, lytic enzymes isolated from Trichoderma harzianum are particularly effective against fungal infection. In particularly preferred embodiments, a β-1,6-glucanase isolated from Trichoderma harzianum is administered to mammals to treat invasive Aspergillus infection.

Such compositions may be formulated so as to be adapted to the specific method of administration. Such compositions may be optimized for administration of the enzyme by parenteral, topical or oral administration. Additionally, the enzyme may be administered by cellular transformation vectors containing nucleic acid sequences encoding therapeutic lytic enzymes.

In a third embodiment, methods for isolating and purifying lytic enzymes from Trichoderms are disclosed. Specifically, the methods are directed to obtaining fungal lytic enzymes with a degree of purity acceptable for therapeutic use in treating fungal infections in human subjects. The methods comprise the steps of (i) precipitating the cellular material from a Trichoderma species, (ii) isolating the proteins therefrom, (iii) precipitating the lytic enzyme by adding its substrate(s), and (iv) purifying the enzyme by isoelectric focusing. The method disclosed herein is applicable to a variety of lytic enzymes from Trichoderma species, and may be practiced on a variety of species to isolate a variety of lytic enzymes without undue experimentation. Exemplary lytic enzymes according to the present invention include glucanases, chitinases, chitosanases and proteases. In preferred embodiments, the present method is applied to isolating and purifying β1-6 glucanase from Trichoderma harzianum.

In a fourth embodiment, the present invention encompasses nucleic acid sequences encoding therapeutic lytic enzymes from Trichoderma species, cellular transformation vectors containing nucleic acid sequences encoding therapeutic lytic enzymes from Trichoderma species, and cells transformed with said cellular transformation vectors. The present invention further provides methods of administering enzymes by genetic therapy techniques wherein a nucleotide encoding the therapeutic compound is administered to a cell or to an organism in order to produce the therapeutic compound endogenously. Those of skill in the art will appreciate many methods for administering transformation vectors containing nucleic acid sequences encoding therapeutic lytic enzymes.

### BRIEF DESCRIPTION OF THE FIGURES

FIG. 1 shows the survival rate after glucanase treatment of Aspergillus infection in mice over a 20-day period of treatment.

FIG. 2 shows the effect of recombinant Trichoderma β-1,6-glucanase on the survival rate of immunosuppressed mice after challenge with Aspergillus.

FIG. 3 illustrates the survival statistics for up to 18 days post inoculation among subjects treated with PBS intraperitoneal, chitinase intraperitoneal at a dosage of 100 mg/kg, chitinase intravenous at a dosage of 100 mg/kg, chitinase intraperitoneal at a dosage of 25 mg/kg, and amphotericin B intraperitoneal at a dosage of 1 mg/kg.

### DETAILED DESCRIPTION OF THE INVENTION

The present invention features methods of treating fungal infections in mammals including humans by administering lytic enzymes and compositions comprising the same. Moreover, the present invention features novel methods for isolating and purifying lytic enzymes from Trichoderms.

The present invention provides novel methods and compositions for treating a variety of fungal and other microbial diseases in mammals including humans by administering one or more lytic enzymes according to the present invention. The present invention differs substantially from many other forms of medical therapy for fungal infections because conventional therapy methods use small molecules that inhibit fungal cell wall and membrane sterol synthesis. Because of the mechanisms of action, the existing methods have significant toxicity and side effects in the recipients. As described above, treatment of fungal infection based on polyenes and azoles is often toxic and has many undesirable side effects. Furthermore, the use of the existing anti-fungal agents has also resulted in the emergence of populations of resistant fungi and new pathogenic fungi strains. Treatment with the new echinocandin antifungal agent Cancidas was associated with adverse effects, including fever, phlebitis/thrombophlebitis and/or infused vein complications, headache, nausea, vomiting, rash, skin flushing and mild liver function test elevations, and one case of anaphylaxis (FDA Talk Paper, Jan. 29, 2001, http://www.fda.gov/opacom/hpnews.html).

The instant invention features a new mechanism for treating fungal infections by administering a pharmaceutically effective amount of one or more lytic enzymes useful to degrade fungal cell walls and to eventually cause fungal cell lysis and death. The instant method is especially designed to target invasive forms of fungal infection for which the existing methods are not optimally effective. As described above, polyenes and azoles have satisfactory effects on common species of fungi such as Candida, Histoplasma and Coccidiodes, but they exhibit little activity against the invasive Aspergillus which are a common cause of death in the immunosuppressed population. In addition, the instant method is also effective in treating fungal infections that are resistant to the existing methods of treatment. In two human subjects infected with pulmonary coccidioidomycosis and one patient with Aspergillus pulmonary and mediastinal infection, mycolases comprising chitinase, β1,3-glucanase, and exo-glycosidases from Physarum polycephalum against Candida pseudotropicalis and Candida albicans administered with amphotericin B were not effective in eliminating the fungal infections (Chalkley, et al., Sabouraudia: J. Med. Vet. Mycol. 23:147-164 (1985)).

In a preferred embodiment, a β1-6 glucanase enzyme isolated from Trichoderma harzianum, shown to possess potent anti-fungal activity in in vitro assays, is administered. As described above, β1,6-Glucans represent only about five percent of the cell wall, but may play a key role by cross-linking the GPI cell wall proteins to β1,3-glucan, and to a lesser extent to chitin. Furthermore, a greater percentage of cell wall proteins become resistant to β1,3-gluanase extraction as a result of increased formation of the chitin→β1,6-glucan linkage. β1,6-glycosylated cell wall proteins have been observed in several other fungi such as the yeast Hansenula polymorpha, the medically important fungi Aspergillus fumigatus, Candida albicans, and Wangiella (Exophiala) dermatitidis, the plant pathogen Fusarium oxysporum, and in the food spoilage fungi Aspergillus niger, Paecilomyces variotti, and Penicillium roquefortii. The β1,6-glucosylated cell wall proteins are widespread in the largest class of fungi, Ascomycetes. (Kapteyn, et al., Biochim. Biophys. Acta 1426: 373-383 (1999)).

As described above, polyenes, azoles, and mycolases containing mixtures of chitinase, β1,3-glucanase, and exo-glycosidases from Physarum polycephalum were ineffective in treating invasive Aspergillus infection (Chalkley, et al., Sabouraudia: J. Med. Vet. Mycol. 23:147-164 (1985)). Thus, in more preferred embodiments, a β1-6 glucanase enzyme isolated from Trichoderma harzianum is administered to effectively treat invasive Aspergillus infection. Unlike prior disclosures, such as International Patent Application No. WO 95/31534 and U.S. Pat. Nos. 5,770,406 and 6,022,723, which disclose a method of using a DNA construct encoding β1,6-endo-glucanase activity in degradation or modification of beta-glucan-containing materials, the fungal lytic enzymes of the present invention are administered directly to a subject in need thereof to exert a therapeutic action in vivo. No studies have shown the therapeutically effective use of the lytic enzymes isolated from Trichoderms, such as β1,6-glucanase or chitobiosidase, for the in vivo treatment of fungal infection, including invasive Aspergillosis.

In another aspect, the present invention also provides for the use of the lytic enzymes for the manufacture of pharmaceutical compositions comprising one or more lytic enzyme(s). Preferably, the pharmaceutical compositions are used or treating fungal infections. Therapeutic enzymes may be administered in a number of ways such as parenteral, topical, intranasal, inhalation or oral administration. In some embodiments, the invention provides for administering the enzyme in a pharmaceutical composition together with a pharmaceutically acceptable carrier, which may be solid, semi-solid or liquid or an ingestible capsule. Examples of pharmaceutical compositions useful in the present invention include tablets and drops, such as nasal drops. Compositions for topical application include, but are not limited to ointments, jellies, creams and suspensions, aerosols for inhalation, nasal spray, and liposomes. One or more lytic enzyme will comprise between 0.05 and 99% or between 0.5 and 99% by weight of the composition. In preferred embodiments, the enzyme content may be between 0.5 and 20% for injection and between 0.1 and 50% for oral administration.

To produce pharmaceutical compositions for oral application containing the therapeutic lytic enzyme(s), the enzyme(s) may be mixed with a solid, pulverulent carrier. The carrier may include, but is not limited to lactose, saccharose, sorbitol, mannitol, a starch (for example, a potato starch or a corn starch), amylopectin, laminaria powder, citrus pulp powder, a cellulose derivative and gelatine. The pharmaceutical compositions may also include lubricants such as magnesium or calcium stearate or a Carbowax or other polyethylene glycol waxes, and they may be compressed to form tablets or cores for dragees. If dragees are required, the cores may be coated with, for example, a concentrated sugar solution. The sugar solutions may contain gum arabic, talc and/or titanium dioxide, or alternatively a film forming agent dissolved in easily volatile organic solvents or mixtures of organic solvents. Dyestuffs may be added to such coatings, for example, to distinguish between different contents of active substance. For a composition of soft gelatine capsules consisting of gelatine, or glycerol as a plasticizer, or similar closed capsules, the active substance may be admixed with a Carbowax® or a suitable oil such as sesame oil, olive oil, or arachis oil. Hard gelatine capsules may contain granulates of the active substance with solid, pulverulent carriers such as lactose, saccharose, sorbitol, mannitol, starches (for example, potato starch, corn starch or amylopectin), cellulose derivatives or gelatine, and they may also include magnesium stearate or stearic acid as lubricants.

Therapeutic lytic enzymes of the present invention may also be administered parenterally such as by subcutaneous, intramuscular or intravenous injection or by sustained release subcutaneous implant. In subcutaneous, intramuscular and intravenous injection, a therapeutic enzyme or other active ingredient may be dissolved or dispersed in a liquid carrier vehicle. For parenteral administration, the active material may be suitably admixed with an acceptable vehicle, preferably of the vegetable oil variety such as peanut oil, cottonseed oil and the like. Other parenteral vehicles such as organic compositions using solketal, glycerol, formal, and aqueous parenteral formulations may also be used. For parenteral application by injection, compositions may comprise an aqueous solution of a water soluble pharmaceutically acceptable salt of the active acids according to the invention, desirably in a concentration of 0.5-10%, and optionally also a stabilizing agent and/or buffer substances in aqueous solution. Dosage units of the solution may advantageously be enclosed in ampoules. When therapeutic enzymes are administered in the form of a subcutaneous implant, the compound may be suspended or dissolved in a slowly dispersed material known to those skilled in the art or administered in a device which slowly releases the active material through the use of a constant driving force such as an osmotic pump. In such cases, administration over an extended period of time may be possible.

For topical application, the pharmaceutical compositions are suitably in the form of an ointment, gel, suspension, cream or the like. The amount of active substance may vary, for example between 0.05-20% by weight of the active substance. Such pharmaceutical compositions for topical application may be prepared in known manners by mixing the active substance with known carrier materials including but not limited to isopropanol, glycerol, paraffin, stearyl alcohol, polyethylene glycol. The pharmaceutically acceptable carrier may also include a known chemical absorption promoter. Examples of absorption promoters are dimethylacetamide (U.S. Pat. No. 3,472,931), trichloro ethanol or trifluoroethanol (U.S. Pat. No. 3,891,757), certain alcohols and mixtures thereof (British Patent No. 1,001,949). A carrier material for topical application to unbroken skin is also described in the British patent specification No. 1,464,975, which discloses a carrier material consisting of a solvent comprising 40 70% (v/v) isopropanol and 0 60% (v/v) glycerol, the balance, if any, being an inert constituent of a diluent not exceeding 40% of the total volume of solvent.

The dosage at which pharmaceutical compositions containing one or more lytic enzymes are administered may vary within a wide range and depends on various factors, such as the severity of the infection and the age of the patient. The dosage may have to be individually adjusted. In preferred embodiments, the amount of therapeutic enzyme is from about 0.1 mg to about 2000 mg or from about 1 mg to about 2000 mg per day. The pharmaceutical compositions containing a therapeutic lytic enzyme may suitably be formulated so that they provide doses within these ranges either as single dosage units or as multiple dosage units. In addition to containing a therapeutic lytic enzyme (or therapeutic lytic enzymes), the pharmaceutical compositions may contain one or more substrates or cofactors for the reaction catalyzed by the therapeutic enzyme in the compositions.

The therapeutic lytic enzymes according to the present invention may be administered by means of transforming patient cells with nucleic acids encoding a therapeutic enzyme when the therapeutic enzyme is a protein or ribonucleic acid sequence. A nucleic acid sequence encoding a therapeutic lytic enzyme may be incorporated into a vector for transformation into cells of a subject to be treated. A vector may be designed to integrate into the chromosomes of the subject, for example, retroviral vectors, or to replicate autonomously in the host cells. Vectors containing nucleotide sequences encoding a therapeutic lytic enzyme may be designed to provide for continuous or regulated expression of the enzyme. Additionally, the genetic vector encoding the therapeutic enzymes may be designed to stably integrate into the cell genome or to only be present transiently. The general methodology of conventional genetic therapy may be applied to polynucleotide sequences encoding therapeutic enzymes. Reviews of conventional genetic therapy techniques can be found in Friedman, Science 244:1275-1281 (1989); Ledley, J. Inherit. Metab. Dis. 13:587-616 (1990); and Tososhev et al., Curr Opinions Biotech. 1:55-61 (1990).

The present invention provides a method for isolating or purifying lytic enzymes from Trichoderms. Specifically, the methods are directed to obtaining fungal lytic enzymes with a degree of purity acceptable for therapeutic use in treating fungal infections in human subjects. The U.S. Food and Drug Administration has issued recommendations for assembling chemical and technological data currently considered appropriate for an enzyme preparation, including guidelines regarding the purity of enzyme preparations (Enzyme Preparations: Chemistry Recommendations for Food Additive and GRAS [Generally Recommended As Safe] Affirmation Petitions, Version 1.1, Jan. 23, 1993; U.S. Food and Drug Administration, Center For Food Safety and Applied Nutrition, Office of Premarket Approval, Chemistry Review Branch). Various studies have shown that impurities, such as anticomplement activity, in protein preparations, including immunoglobulin preparations, may be associated with the development of allergic and anaphylactic reactions (Lundblad, et al, Rev. Infect. Dis. 8 (Suppl. 4):S382-90 (1986); Scheiermann and Kuwert, Dev. Biol. Stand. 44:165-171 (1979)). Furthermore, impurities may be associated with unwanted biological activities and interference with desired therapeutic effects. Thus, enhanced purity of protein preparations would contribute to greater efficacy of the therapeutic protein (Ueshima, et al., J. Clin. Hosp. Pharm. 10(2): 193-202 (1985); Ehrlich, et al., Clin. Chem. 34(9): 1681-8 (1988)). The method comprises the steps of (i) precipitating the cellular material from a Trichoderma species, (ii) isolating the proteins therefrom, (iii) precipitating the enzyme by addition of its substrate(s), and (iv) purifying the enzyme by isoelectric focusing. The lytic enzymes that may be isolated and purified by the present invention include glucanases, chitinases, chitosanases and proteases. Exemplary lytic enzymes include, but are not limited to endo and exo (1,3)-β-glucanases, endo and exo (1,4)-β-glucanases, endo and exo (1,2)-β-glucanases, endo and exo (1,6)-β-glucanases, endochitinases, exochitinases, chitobiohydrolases, endochitosanases, exochitosanases, 1,4-β-poly-N-acetyl-D-glucosaminidase, and endo and exo 1,4-β-poly-D-glucosaminidase. The invention is also specifically intended to encompass lytic enzymes from Trichoderms such as, but not limited to, T. atroviride, T. cirtinoviride, T. hamatum, T. harzianum, T. koningii, T. lignorum, T. longibrachiatum, T. polysporum, T. pseudokoningii, T. reesei, T. saturnisporum, T. todica, T. virgatum and T. viride. In some preferred embodiments, the present method has been successfully applied to isolating and purifying a β1-6 glucanase enzyme from Trichoderma harzianum. This particular enzyme has a molecular weight of about 43,000 daltons and an isoelectric point of about 5.8.

### EXAMPLES OF THE PREFERRED EMBODIMENTS

The following examples further illustrate the present invention. These examples are intended merely to be illustrative and are not to be construed as limiting.

### Example 1

### Isolation of β1-6 Glucanase from Trichoderma harzianum

Procedure. Culture of Trichoderma harzianum for enzyme isolation was accomplished under the following growth conditions. T. harzianum (ATCC 52324) was obtained from the American Type Culture Collection (Rockville, Md.). The lyophilized pellet was resuspended in modified Czapek medium (250 ml containing 0.2 mg/L MgSO₄.7H₂0, 0.9 mg/L KH₂PO₄, 0.2 mg/L KCl, 1.0 mg/L NH₄NO₃, and 0.002 mg/L Zn⁺⁺) supplemented with 10% glucose. The culture was allowed to grow for 48 hours at 24° C. with aeration. The culture was filtered, and the cells were resuspended in the media described above (1.0 L) except that 1.5% chitin was substituted for the 10% glucose. The culture was incubated for four days at 24° C. with aeration.

Isolation of lytic enzymes was performed at 4° C. Following the incubation, the cells were filtered through a filter paper (Whatman no. 1) and the filtrate was centrifuged at 6,000×g for 10 minutes. The supernatant was precipitated with ammonium sulfate to 80% saturation. The precipitate was recovered by centrifugation at 12,000×g for 20 minutes and resuspended in distilled water. The mixture was then dialyzed against 50 mM potassium acetate buffer, pH 5.5. The dialyzed fraction contained lytic enzymes.

The crude enzyme (10 g) was dissolved in water (100 ml) and dialyzed against sodium acetate buffer, pH 5.0. The dialyzed enzyme was adsorbed on alcohol precipitated pustulan (β1-6 glucan, 5.0 g) at 4° C. for 20 minutes. The supernatant containing non-adsorbed enzyme was collected by centrifugation and readsorbed on fresh pustulan. The process was repeated for three times. All pustulan-enzyme precipitates were pooled and washed three times with sodium acetate buffer (pH 5.0, 100 ml) containing 1M sodium chloride. The pustulan-enzyme complex was then resuspended in phenylmethylsulfonyl fluoride (1 mM) with 0.02% sodium azide and incubated overnight at 37° C. to digest and release the enzyme. All clarified supernatants obtained after pustulan-enzyme incubation treatment were pooled and centrifuged at 12,000×g for 10 minutes. The supernatant was dialyzed against sodium acetate buffer, pH 5.0. This final preparation was then subjected to preparative isoelectric focusing using ampholytes from pH 5-7 in a Rotofor® unit. Isolecectric focusing was run at 12 watts at 4° C. for 3 hours. Fractions were collected, pooled, concentrated and dialyzed. Purified enzyme was used for physicochemical characterization and anti-fungal testing.

Results. The final preparation product, β1-6 Glucanase, appeared as a single band on sodium dodecyl sulfate polyacrylamide gel electrophoresis (SDS-PAGE) with an apparent molecular weight of 43,000 daltons and an isolelectric point of 5.8. The purified enzyme had a pH optima of 5.0 and temperature optima of 40-50° C. The enzyme was highly specific in cleaving only β1-6 linked polymers of glucose as demonstrated in Example 2 below producing β1-6 polymers of glucose of greater than 2 units from pustulan.

### Example 2

### Characterization of Specific β1-6 Glucanase Enzyme Activity

Procedure. The activity of β1-6 glucanase was determined by incubating the enzyme (0.3 ml) in 1% pustulan in 50 mM sodium acetate buffer (pH 5.0, 0.1 ml) for 10 minutes at 37° C. The reaction was stopped by adding dinitrosalicylic acid (0.75 ml). The reducing power of the digest was measured. One unit of enzyme is the amount of enzyme that forms an increase of reductive power equivalent to 1 μm of glucose per minute.

Results. The crude enzyme was found to contain 2 units per milligram of protein. The final purified enzyme was found to contain 20 units per milligram of protein, or approximately 100-fold purification.

### Example 3

### Characterization of Anti-Fungal Activity

Procedure. The anti-fungal activity of the β1-6 glucanase was determined in 96-well microtiter plates using RPMI-1640 medium with glutamine (150 ml) and a spore suspension of approximately 1000 spores of Aspergillus fumigatus in the same RPMI-1640-glutamine medium (50 ml). β1-6 glucanase enzyme (0.008-0.08 units) in 50 mM sodium acetate buffer (pH 5.0) was added. Control buffer without enzyme was also used. The microtiter plate was incubated at room temperature for 18 hours and then transferred to a 37° C. incubator. Growth of fungal hyphae was monitored using an inverted microscope.

Results. The control wells in the plate had good growth of hyphae with over 50 hyphal colonies per well. The crude enzyme preparation was similar to the control, but the purified enzyme showed only minimal hyphal growth with fewer than 5 hyphal colonies per well.

### Example 4

### Characterization of Anti-Fungal Activity in Microorganisms and Animals

Procedure. Fungal Organisms. A. fumigatus phialoconidia (conidia) were used as infectious particles throughout this study. Isolates of A. fumigatus originally obtained from patients were maintained on potato dextrose agar for spore and conidia harvesting. Spores or conidia were harvested in saline and vortex-mixed to break up clumps. The mixture was filtered through eight layers of cheesecloth and washed three times in saline. The concentrate was examined by light microscopy. Spore suspensions were free of hyphal fragments. Viability counts for the production of inocula were determined on Sabouraud's agar. The viability of spores or conidia was always >95%.

Induction of Immunodeficiency and Cortisone Acetate Treatment. Four- to six-week-old female pathogen-free mice (CD-1 strain) were obtained from Charles River Breeding Laboratories (Kingston, N.Y.). The mice were given free access to water and a standard laboratory diet until 8 hr before cyclophosphamide or buffer injection, when food was withdrawn. Cyclophosphamide was used to induce immune suppression. Briefly, cyclophosphamide was dissolved in ice-cold citrate buffer (pH 4.2). A dose of 250 mg/kg (0.2 ml) was injected intraperitoneally within 10 min of dissolution. Control animals received buffer (0.2 ml). Mice were used in the experiment 7-14 days after cyclophosphamide or buffer injection. Cortisone acetate was injected subcutaneously in a daily dose of 125 mg/kg in 0.15 M NaCl solution (0.1 ml) for six consecutive days just before challenge. Control animals received NaCl solution alone (0.1 ml).

Animal Models. Graded doses (100 to 10 million) of spores, conidia, or sterile aqueous inocula were administered intravenously seven days after the injection of cyclophosphamide or buffer or on the day after completion of the cortisone acetate or buffer treatment. Animals were observed for 15 days and the LD 50 determined. When the animals died or were killed, the organ distribution of viable fungi was determined. Portions of lung tissue were processed and stained with Grocott methenamine silver and hematoxylin and eosin for histological evaluation (Waldorf et al., J Infectious Disease 150:752-760, 1984).

Enzyme Treatment. Thirty minutes after intravenous inoculation with spores or conidia, animals were administered intravenously either normal saline (0.1 ml) as controls or a β1-6 glucanase solution (0.15 ml, 2 units of activity). Treatment continued every 24 hours for 5 days.

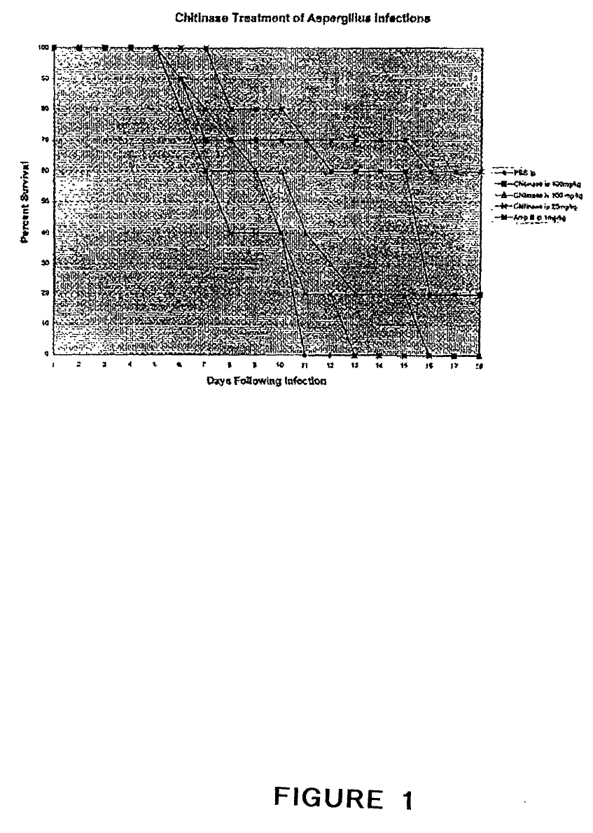

Results. There were 8 control animals and 5 enzyme-treated animals. The survival curve is illustrated in FIG. 1. The test of significance for the result was a p value of <0.05 (2-tailed Wilcoxon Rank-Sum analysis). The results showed 60% of mice treated with β1-6 glucanase were surviving 15 to 20 days post inoculation. None of the untreated mice were surviving at the same time.

### Example 5

### Effect of Trichoderma β-1,6-glucanase on the survival rate of immunosuppressed mice after challenge with Aspergillus

Animals were treated as described in Example 4 except with the following changes in protocol. There were ten mice in each of the four experimental groups, designated Control, Glucanase (50 mg/kg), Glucanase (25 mg/kg), and Glucanase (10 mg/kg). Cyclophosphamide was administered at a dose of 200 mg/kg IP three days before infection, on the day of infection (Day 0) and on Day 3 after infection. The calculated challenge dose of Aspergillus fumigatus conidia was 2.5×10⁷/mouse, administered on Day 0. Antifungal therapy was started on day 1, wherein doses of 50, 25 and 10 mg/kg Trichoderma β-1,6-glucanase were administered intraperitoneally each day.

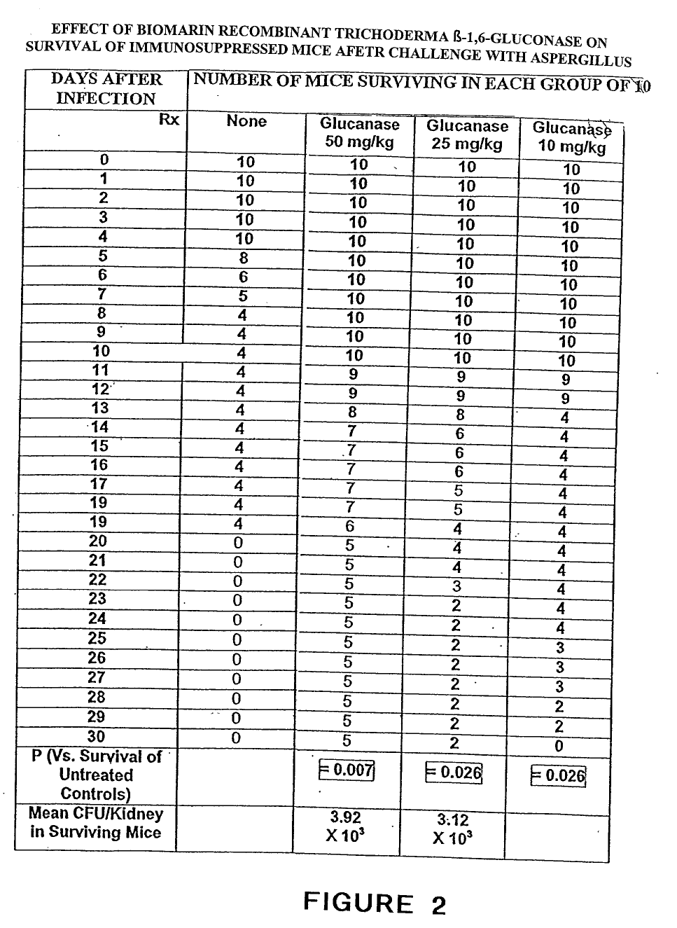

Results. As shown in FIG. 2, Trichoderma β-1,6-glucanase caused a significant increase in survival rate of mice infected with Aspergillus fumigatus conidia at doses of 50, 25 and 10 mg/kg Trichoderma β-1,6-glucanase administered intraperitoneally each day. Increasing doses of the enzymes were associated with enhancement of survival rate, consistent with dose-related efficacy of Trichoderma β-1,6-glucanase. These results are consistent with a dose-related efficacy of this lytic enzyme. There was no evidence of glucanase toxicity.

### Example 6

### Isolation of Chitinase from Trichoderma harzianum

Procedure. Fungal organisms. Trichoderma harzianum phialoconidia (conidia) were used as infectious particles. Isolates of A. fumigatus originally obtained from patients were maintained on potato dextrose agar for spore and conidia harvesting. Spores of conidia were harvested in saline, vortex mixed to break up clumps, filtered through eight layers of cheesecloth, washed three times in saline, and examined by light microscopy. Spore suspensions were free of hyphal fragments. Viability counts for the production of inocula were determined on Sabouraud's agar. The viability of spores or conidia was always >95%.

Enzyme production and purification. Enzymes were produced using strain PI of Trichoderma harzianum (ATCC 74058). The strain was grown for 4 days on a rotary shaker in Richard's modified medium, which contained 10 g of KNO₃, 5 g of KH₂PO₄, 2.5 g of MgSO₄-7H₂O 2 mg of FcCI₃, 1% (w/v) crab shell chitin (Sigma), 1% polyvinylpyrrolidone (Polyclar AT, GAF Corp., Wayne, N.J.), 150 ml of V8 juice, and 1,000 ml of H₂O at pH 6.0. The biomass was removed by filtration, the supernatant dialyzed against 50 mM potassium phosphate buffer (pH 6.7), and enzymes separated by gel filtration chromatography in a chromatography column packed with Sephacryl S-300 (Pharmacia LKB Biotechnology, Upsala, Sweden), followed by chromatofocusing. A single protein with endochitinase activity was obtained. Purity was confirmed by using native and sodium dodecyl sulfate (SDS) polyacrylamide gel electrophoresis (PAGE) (PhastSystem, Pharmacia) and by isoelectric focusing (IEF). The fractions containing chitobiosidase activity were further separated in a Rotofor IEF cell (Bio-Rad, Richmond, Calif.). Peak fractions containing only chitobiosidase activity were collected, dialyzed against distilled water, and concentrated to dryness in a SpeedVac apparatus (Savant Instruments, Farmingdale, N.Y.). PAGE, followed by staining, with Coomassie blue, indicated a single protein band. Protein concentration in the enzyme preparations was determined using the Micro BCA protein assay (Pierce, Rockford, Ill.,) with trypsin inhibitor from soybean (Sigma) as the standard protein. Enzyme solutions were kept at 4° C. and utilized for the biossays within 2 weeks or dried in a SpeedVac apparatus and stored at −20° C. until used.

Induction of immunodeficiency and cortisone acetate treatment. Four- to six-week-old female pathogen-free mice (CD-1 strain) were obtained from Charles river Breeding Laboratories (Kingston, N.Y.). The mice were given free access to water and standard laboratory diet until 8 hours before cyclophosphamide or buffer injection, when food was withdrawn. Cyclophosphamide was used to induce immune suppression. Cyclophosphamide was dissolved in ice-cold citrate buffer (pH 4.2), and a dose of 250 mg/kg in 0.2 ml was injected intraperitoneally within 10 minutes of dissolution. Control animals received 0.2 ml of buffer. Mice were used in the experiment 7-14 days after cyclophosphamide or buffer injection. Cortisone acetate was injected subcutaneously in a daily dose of 125 mg/kg in 0.1 ml of 0.15 M NaCl solution for six consecutive days just before challenge. Control animals received 0.1 ml of NaCl solution alone.

Animal Model. Graded doses (100 to 10 million) of spores, conidia, or sterile aqueous inocula were administered intravenously seven days after the injection of cyclophosphamide or buffer or on the day after completion of the cortisone acetate or buffer treatment. Animals were observed for 15 days, and the LD 50 was determined. When the animals died or were killed, the organ distribution of viable fungi was determined. Portions of lung tissue were processed and stained with Grocott methenamine silver and hematoxylin and eosin for histological evaluation (Waldorf et al., J Infectious Disease 150:752-760, 1984).

Enzyme Treatment. Thirty minutes after intravenous inoculation with spores or conidia, animals were administered intravenously either 0.1 ml of normal saline (controls) or 0.15 ml containing 2 units of chitinase. Treatment continued every 24 hours for 5 days. In all experiments, treatment starts 30 min after infection and daily thereafter until mice are dead or moribund (or until untreated controls are all dead or moribund).

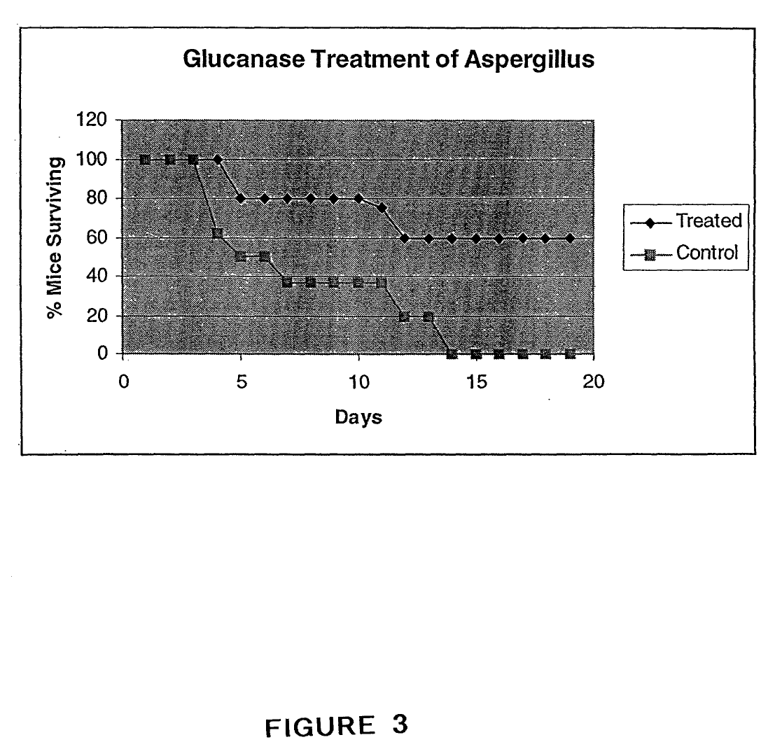

Results. The survival statistics for 18 days post inoculation among subjects treated with PBS intraperitoneal, chitinase intraperitoneal at a dosage of 100 mg/kg, chitinase intravenous at a dosage of 100 mg/kg, chitinase intraperitoneal at a dosage of 25 mg/kg, and amphotericin B intraperitoneal at a dosage of 1 mg/kg are presented graphically in FIG. 3.

### Example 7

A chitinase molecule of 389 amino acids and a molecular weight of 42,393 having an isoelectric point of 6.3 was produced in an Escherichia coli cell line as described in Invitrogen's catalog (1999 edition, page 191). The expression vector pBAD/His A with cloning sites Nco-Pst having a gene encoding a chitinase was inserted into the E. coli cells.

Fermentation. The chitinase strain is transferred from a previous LB positive ampicillin (100 μg/ml) plate or a frozen stock tube to a new LB positive ampicillin plate. The plate is incubated at 37° C. for 48 hours. Inoculation flasks containing 200 ml of 25 g/l LB media and 0.2% w/v glucose are prepared. Ampicillin (100 μg/ml) is filter sterilized into the flask before inoculation. The shake flask is inoculated with a single colony and incubated for 12 hours. 375 grams of LB media and 30 grams of glucose (dextrose) are dissolved into 4 liters of water. The media is added to a fermentor and brought up to 14 liters. One ml of antifoam is added. The media is sterilized for 35 minutes at 121° C. Once the temperature is stabilized at 37° C., ampicillin is filtered in to achieve 100 μg/ml. The fermentor conditions are set to a pH of 7.0, agitation to 500 rpm, air flow to 15 lpm, temperature at 37° C., back pressure to 2.5 psig, and dissolved oxygen cascade set to 30% before inoculation. The fermentor is inoculated with 200 ml of shake flask culture. The culture is allowed to grow until the glucose is exhausted (about 5 hours). The culture is induced by filter sterilizing in 3 grams of (L) arabinose and maintained at 37° C. for three hours. The temperature is changed to 28° C. overnight. The fermentor is harvested the next morning for a total induction time of 18 hours and a total run time of about 24 hours. The culture is harvested and run through a continuous tubular bowl centrifuge at 230 ml/min to pellet the cells. The cell pellet is stored in a −45° C. freezer. A cell pellet is broken by vortexing the cells with silica and creating shear. The slurry is centrifuged and the supernatant assayed. The average chitinase activity is 4 units per ml.

Purification. Frozen cells are thawed overnight at 4° C. For every gram of cell paste an equal volume of lysis buffer which contains sodium phosphate, pH of 7.4., 100 mM EDTA and 1 μM PMSF is used to resuspend the cell mass. The suspension is passed twice through a homogenizer at 12,000 to 15,000 PSI. Lysed cells are centrifuged for one hour at 19,000×g. This removes cell debris and retains greater than 95% of enzyme activity. The lysate is readjusted to a pH of 7.4. A Q-Sepharose Fast Flow, 1.5 L column is used as a flow through step. The column is equilibrated with sodium phosphate, pH of 7.4. The flow rate for Q-Sepharose is done at 100 cm/hour. Conductivity of the cell lysate should be maintained as low as possible, less than 10 mS/cm. Chitinase does not bind to the media if the pH is controlled properly. Material from only the first flow through peak is collected and used for the next step. A Phenyl-Sepharose Fast Flow, 3-4 L column is used to capture chitinase. The flow rate for Phenyl-Sepharose is set at 100 cm/hour. Add enough NaCl to the flow through material from the previous step to make the final concentration 3M. Bound chitinase is eluted from the Phenyl-Sepharose column by applying a descending linear salt gradient to 0 M NaCl using 10 column volumes. The fractions comprising the activity are pooled and diafiltered into 25 mM sodium borate, pH 9.0 using a Sartorius Sartocon 10 Kda NMWCO filtration system. The volume of borate buffer used for the diafiltration is about 5 volumes. A DEAE-Sepharose Fast Flow (50 mm diameter×100 mm height) column is used to bind and concentrate chitinase. The flow rate for DEAE-Sepharose is performed at 100 cm/hour. The bound chitinase is eluted off the DEAE-Sepharose column by applying an ascending linear salt gradient to 2 M NaCl. The cleanest fraction is analyzed by SDS-PAGE, which generally comprise the largest amount of activity, are pooled, diafiltered into phosphate buffered saline at pH of 7.4. The usable fractions are pooled and diafiltered into PBS at pH of 7.4 using a sartorius Sartocon 10 Kda NMWCO filtration system. The volume of PBS used for the diafiltration is more than five volumes. The resulting material is assayed for activity, protein, purity and endotoxin.

Fluorescence Assay to measure Chitinase Activity. Chitinase samples reacts with the substrate, 4-Methylumbellifery-N,N′,N″-triacetyl-β-chitotrioside, by cutting off the 4-methyllumbelliferone. This release of 4-methylumbelliferone is measured in microplates in a fluorescence microplate reader. The wavelength of the light for excitation is 355 nm and for emission is 460 nm.

Reagents Preparation. Prepare 0.5 M Sodium Phosphate (500 mL): Add 35.49 g of Sodium Phosphate into 500 mL of water. Mix until clear. (Various volumes can be made according to need.). Prepare 0.5 M Sodium Phosphate Monobasic (500 mL). Add 34.50 g of Sodium monobasic into 500 mL of water. Mix until clear. (Various volumes can be made according to need.)

Prepare 50 mM Phosphate buffer, pH 7.2: Make 1:10 dilution of 0.5 M Sodium Phosphate solution to get a final concentration of 50 mM. Make 1:10 dilution of 0.5 M Sodium Phosphate Monobasic solution to get a final concentration of 50 mM. Use the following ratio to get pH 7.2, approximately 68.4% Na₂HPO₄+31.6% NaH₂PO₄. Use the pH meter to read pH and adjust until pH is at 7.2.

Prepare 1 mM 4-Methylumbellifery-N,N′,N″-triacetyl-β-chitotrioside: Pipette 800 μl of DMSO into vial and mix until clear. Add the 800 μl of 4-Methylumbellifery-N,N′,N″-triacetyl-β-chitotrioside to 5560 μl of 50 mM Phosphate buffer, pH 7.2 to obtain a final volume of 6360 μl.

Assay method. Cracking the cells (if necessary): Pipette 1 ml of sample into 1.5 mL microcentrifuge tube. Centrifuge for 2 min. at 14,000 rpm (revolution per minute). Pour out supernatant from tube. Add 100 μl 50 mM Phosphate buffer, pH 7.2 into tube. Vortex to resuspend pellet into solution. Add approximately 100 μl of glass beads and vortex for 30 sec. and ice for 30 sec. Repeat 6 times. Centrifuge tube for 2 min. at 14,000 rpm. Pipette out supernatant into separate tube.

Prepare 1 mM 4-Methylumbelliferone stock solution: Weigh out 0.0881 g 4-Methylumbelliferone into a tube. Add 25 ml of 50 mM Phosphate buffer, pH 7.2. Mix. Add 6N NaOH into solution until all of the 4-Methylumbelliferone is dissolved. Q.S. solution to 500 mL with 50 mM phosphate buffer, pH 7.2.

Prepare 4-Methylumbelliferone standard curve: This solution can be kept up to twelve months at −20° C.

| 4-Methylumbelliferone | 4-Methylumbelliferone | 50 mM |
|---|---|---|
| Concentration | Stock Solution | Phosphate buffer, |
| μM | 1 mM | pH 7.2 |
| 0 | 0 μl | 10000 μl |
| 1 | 10 μl | 9990 μl |
| 5 | 50 μl | 9950 μl |
| 10 | 100 μl | 9900 μl |
| 15 | 150 μl | 9850 μl |

Reaction Method. Pre-cool plate and all reagents on ice to minimize variability. Pipette 100 μl of each 4-Methylumbelliferone standard (0, 1, 5, 10, 15 μM) into each well. Pipette 95 μl of 50 mM Phosphate buffer, pH 7.2. This will serve as the blank. Pipette 90 μl of 50 Phosphate buffer, pH 7.2 into each well for each sample. Pipette 5 μl of sample into each sample well. Sample(s) may need to be diluted to give a value (μM) between 0-15 μM. Pipette 5 μl of 1 mM 4-Methylumbellifery-N,N′,N″-triacetyl-β-chitotrioside into each sample and the blank. Incubate plate for 10 min. at 37° C. Shake to ensure mixing.

Absorbance reading. Prepare the Fluorescence microplate reader for measurement at: Excitation: 355 nm, Emission: 460 nm. Allow for machine to warm up for at least 30 min. Set up the template to assign standards, samples, and dilution factors as needed. Read plate.

CALCULATIONS. Use the linear regression method available on Microsoft Excel (or equivalent statistical software program) to generate an equation by plotting the absorbance values for each 4-Methylumbelliferone standard (ordinate scale) versus the amount of 4-Methylumbelliferone, i.e., 0, 1, 5, 10, 15 μM (abscissa scale). Record the slope (m), intercept (b), and correlation coefficient (r²) of the linear regression equation (y=mx+b), as determined by the Microsoft Excel software (or equivalent statistical software program).

Calculate and report the amount of each sample, μM, by substituting the absorbance value of each sample into the linear regression equation.

$$Protein Amount ( µ M ) = \frac{Absorbance Value - Intercept ( b )}{Slope ( m )} \times Dilution factor$$

Record all information and calculations for sample(s). Most microplate reader software should automatically do the above calculations. Use the above steps if the microplate reader does not automatically generate the desired values.

Calculate activity units using the following calculation:

$$Activity = \frac{( Protein Amount ( µM ) ) \times ( volume of standard ( L ) )}{( Enzyme vol in rxn ( mL ) ) \times ( rxn time ( min ) )} = units \text{/} mL$$

One unit is defined as the amount of enzyme required to release 1 μmole of methylumbelliferone from MUF-(GlcNAc)₃ per min. at 37° C., pH 7.2. If the Protein Amount (μM) is <1 μM, print result as “<0.002 units/mL”

The acceptance criteria is generally that the coefficient of correlation, r, for the individual absorbance of the standards vs. their respective 4-Methylumbelliferone concentration should be ≧0.980.

### Example 8

The glucanase molecule is 452 amino acids in length with a molecular weight of about 38,000 and an isoelectric point of about 5.8. A gene encoding glucanase was transferred into a Pichia Pastoris cell line mk71 using a pPICZb-Glucl expression vector obtained from Invitrogen.

Inoculum Preparation. About 100 μl of a Gluc 16 glycerol stock was inoculated into 10 ml of BMGY medium. This culture was grown 24 hours and became visibly dense. The 10 ml culture was used to inoculate 200 ml of a batch medium containing 25 g/l sodium hexametaphosphate (EM Science), 34 g/l fermentation basal salts (Invitrogen), 9 g/l ammonium sulfate, 40 g/l glycerol, 4.35 ml/l PTM₁ trace metals (Invitrogen) and histidine (Sigma) supplemented to 0.1% final concentration as needed and grown overnight (16 hours). A three liter fermenter was inoculated with the entire 200 ml flask culture and grown dense with glycerol fed-batch. This three liter fermenter culture was used to seed a 100 liter bioreactor.

Fermenter Preparation. The fermenter was sterilized with 60 liters containing the fermentation basal salts, ammonium sulfate, and glycerol. Sodium hexametaphosphate and PTM1 trace metals were made us as a 10×stock solution, filter sterilized and added to the ferementer after it had been cooled to 30° C. The pH of the medium was adjusted to 5.0 with concentrated ammonium hydroxide for the initial batch culture. A 2500 ml stock solution of 12% (w/v) histidine was prepared and sterilized. 500 ml of histidine stock solution (0.1% final concentration ) was added to the fermenter before inoculation and four more times during the fermentation. The dO₂ and pH probes were calibrated and checked for proper operation. Dissolved oxygen concentration was maintained by varying the agitation between 150 and 500 rpm. When the agitation neared its maximum value, back pressure on the fermenter was increased up to 12 psig to achieve higher oxygen transfer rates. To maintain dO₂ levels at maximum agitation and back pressure, oxygen was supplemented into the air sparge using a mass flow controller. Control of the pH was achieved by ammonium hydroxide addition. Foam control was achieved by automatic addition of KFO 673 (50% solution in methanol) antifoam.

Fermentation and Sampling. Standard Pichia pastoris fermentation protocols were followed for the fermentation. After an initial batch phase growth on glycerol, a glycerol fed-batch was started. The fermentation was fed 20 liters of 50% glycerol before induction. dO₂ spike tests were performed throughout the glycerol fed-batch and each gave a spike time of less than 60 seconds. During the glycerol fed-batch, the pH setpoint was changed to reach the desired induction pH (3.0) by the cultures own natural acidification. Mixed feed (0.2% methanol, 9 ml/1/hr 50% glycerol) Muts induction was started immediately after the end of the glycerol fed-batch. The methanol concentration was controlled using a Raven Biotech controller.

The fermentation trend graphs included with this report show the dissolved oxygen, pH, temperature, and agitation rate. Five samples of the fermentation were taken throughout the time course and numbered consecutively. Three samples were collected pre-induction and two during methanol fed-batch (post-induction). The wet cell weights for samples 1 though 5, respectively, were: 146, 200, 280, and 284 g/l. Sample one and two were taken in the middle of glycerol fed-batch, sample three was taken at the end of glycerol fed-batch, sample four was taken 12 hours post induction, and sample five was taken 20 hours post induction. The cell pellets from the samples were frozen at −20° C.

Histidine additions were made approximately every 100 g/l. The additions were made when the culture WCW was 54 (glycerol batch phase), 200 (glycerol fed-batch phase), and 280 (glycerol fed-batch phase). Finally, a histidine addition was don 12 hours post induction (again, 280 g/l WCW).

The fermentation was harvested by tangential flow microfiltration (0.2 μm PES membrane). The cell suspension was concentrated to 514 g/l (sample 6), about 50 liters, then a constant volume washing technique was used to remove the spent medium from the cell product. The cell suspension was then concentrated to about 40 liters of cell suspension (550 g/l WCW) before snap freezing with liquid nitrogen.

Purification. Cells from 100 L fermentation grown by Invitrogen were concentrated and diafiltered by tangential flow filtration into 25 mM NaOAc, pH 4.5, 1 mM EDTA. The final volume was 40 L with a density of 550 g/l (wet cell weight). The cells were exuded directly into liquid nitrogen and stored frozen at −80° C. Approximately 20 L of cells were passed two times through an Avestin C-50 homogenizer at 30,000 psi at 4° C. and the homogenate was frozen on dry ice, then stored at 80° C. The homogenate was cleared by centrifugation (10,000 rpm, 60 minutes, 4° C.), yielding 13.5 L of cleared lysate containing approximately 35 g of total protein.

The cleared homogenate was adjusted to IM (NH₄)₂SO₄ by adding one volume of 25 mM NaOAc, pH 4.5, 3M (NH₄)₂SO₄, 1 mM EDTA per two volumes of lysate. The homogenate was run on a 1.3 L Phenyl Sepharose Fast Flow (Pharmacia Catalog #17-0965-03) 14 cm diameter×8.5 cm height column at a flow rate of 300 ml/min. The column was preconditioned with 5 column volumes of 0.1 N NaOK, 5 column volumes of water, and 5 column volumes of EQ buffer ( 25 mM NaOAc, pH 4.5, 1M (NH₄)₂SO₄, 1 mM EDTA. A 10 column volume wash of EQ buffer and a ten column volume wash of 25 mM NaOAc, pH 4.5, 0.5 M (NH₄)₂SO₄, 1 mM EDTA was performed. Elution was done with ten column volumes of 25 mM NaOAc, pH 4.5, 1 mM EDTA.

Glucanase assay. Protein concentrations were determined by Bradford assay using Bovine Gamma Globulin as a standard. Activity was determined by reacting glucanase containing solutions with the fluorogenic substrate GT-4-mu (4-methylumbelliferyl β D-gentotrioside; Toronto Research Chemicals Inc. Catalog #M33449). The activity assay was performed as follows: Made two fold serial dilution of protein in eight wells of a 96 well microtiter plate (10 μl of Elution Buffer). Initiate the reaction by adding 100 μl of prewarmed (about 37° C.) 40 μM GT-4-Mu (in 50 mM NaOAc, pH 5.5). Determine the RFU's in 5 minute rate assay at 37° C., λ_{EX}=355, λ_{EM}=460. Calculate RFU's/ml from RFU readings in the linear range of the dilution series (RFU×dilution factor/0.01 ml). Total reactivity (RFU)=RFU/ml×volume. Values greater than 10,000 are considered good.

### Example 9

The glucanase molecule is 452 amino acids in length with a molecular weight of about 38,000 and an isoelectric point of about 5.8. A gene encoding glucanase was transferred into a Saccharomyces cerevisiae using a pYES2 expression vector having cloning sites sac-xho.

The Saccharomyces cerevisiae strain is transferred from a previous yeast nitrogen base (YNB) without amino acids plus URS drop out plate to a new plate and incubated at 30° C. for 48 hours. Inoculation flasks containing 200 ml of 6.7 g/l YNB without amino acids and 0.77 g/l URA drop out (Clontech) media are prepared. Shake flasks are inoculated with a single colony and incubated for 12 hours. 94 g of YNB without amino acids and 11 g of URA drop out powder was dissolved into 4 L of water. Media was added to the fermentor and brought up to 14 L in the fermentor. One ml of antifoam was added. The media was sterilized for 35 minutes at 121° C. Once the temperature was stabilized at 30° C., 14 g of sterile glucose was added. Fermentor conditions were controlled at pH 6.5, agitation 500 rpm, air flow 15 lpm, temperature 30° C., back pressure 2.5 psig. The fermentor was inoculated with 200 mls of shake flask culture. Once the glucose concentration is exhausted (around 32 hours), the culture is induced with 300 grams of Galactose. The culture remains at 30° C. and 1 vvm of air for another 36 hours. The cell broth is run through a continuous centrifuge to pellet the cells. The cells are stored in a −80° C. freezer. The Glucanase assay is read by fluorescent spectrophotometry and good levels of expression are considered to be greater than 10,000 RFU/microgram of protein. The activity of the glucanase may be assessed as per the protocol set forth in the preceding example.

The foregoing written specification is considered to be sufficient to enable one skilled in the art to practice the invention. Indeed, modifications of the modes described for carrying out the invention which are obvious to those skilled in the pharmaceutical arts or related fields are intended to be within the scope of the following claims.

We claim:

1. A method of treating a fungal infection comprising the steps of:
(a) extracting lytic enzymes from a fungus;
(b) purifying said fungal lytic enzymes to a degree of purity acceptable for therapeutic use in human subjects;
(c) formulating said purified fungal lytic enzymes in a pharmaceutical composition together with a pharmaceutically acceptable carrier; and
(d) administering said pharmaceutical composition to a subject in need thereof an amount therapeutically effective to treat said fungal infection.

2. The method according to claim 1, wherein the lytic enzyme is selected from the group consisting of Endo-(1-3)-β-N-glucanase, Exo-(1-3)-β-N-glucanase, Endo-(1-6)-β-N-glucanase, Exo-(1-6)-β-N--glucanase, Endo-(1-4)-p-β-glucanase, Endo-(1-4)-β-N-glucanase, Endo-(1-2)-β-N-glucanase, Endo-(1-2)-β-N-glucanase, 1-4-β-poly-N-acetyl-D-glucosaminidase, a chitinase, a chitobiosidase, a chitobiohydrolase, endo-1 -4-β-poly-D-glucosaminidase, exo-1-4-β-poly-D-glucosaminidase, and a protease.

3. The method according to claim 1, wherein said fungal lytic enzyme is a β1-6 glucanase.

4. The method according to claim 1, wherein said lytic enzyme comprises between about 0.05% and about 99% by weight of said pharmaceutical composition.

5. The method according to claim 1, wherein said lytic enzyme comprises between about 0.5% and about 99% by weight of said pharmaceutical composition.

6. The method according to claim 1, wherein said therapeutically effective amount of said lytic enzyme is between about 0.1 milligram and about 2000 milligrams per day.

7. The method according to claim 6, wherein said therapeutically effective amount of said lytic enzyme is between about 1 milligram and 2000 milligrams per day.

8. The method according to claim 1, wherein said fungus is Trichoderma.

9. The method according to claim 8, wherein said fungus is Trichoderma harzianum.

10. The method according to claim 1, wherein the fungal infection is caused by a species having a cell wall comprising a β-1,6-glucan.

11. The method according to claim 1, wherein said fungal infection is caused by a species of Aspergillus.

12. The method according to claim 11, wherein said fungal infection is caused by Aspergillus fumigatus.

13. The method according to claim 1, wherein said fungal infection is invasive aspergillosis.

14. The method according to claim 1, wherein said is selected from the group consisting of (a) administrating topical via a carrier material selected from a group consisting of isopropaol, glycerol, paraffin, stearyl alcohol, and polyethylene glycol; (b) parenteral administering; (c) intranasal administering; (d) administering via inhalation; and (e) oral administrating.

15. The method according to claim 14, wherein said parenteral administration of said lytic enzyme is accomplished by subcutaneous, intramuscular and intravenous injection or by sustained release subcutaneous implant, such that a therapeutically effective amount of said lytic enzyme contacts the sites of fungal infection in vivo via systemic absorption and circulation.

16. The method according to claim 1, wherein said lytic enzyme comprises between about 0.5% and about 20% by weight of said pharmaceutical composition for administration by injection.

17. The method according to claim 1, wherein said lytic enzyme comprises between about 0.1% and about 50% by weight of said pharmaceutical composition for oral administration.

18. The method according to claim 1, wherein said fungal lytic enzyme or a biologically active fragment thereof is encoded by a nucleic acid sequence contained within a recombinant plasmid.

19. The method of claim 18, wherein said recombinant plasmid is administered to a cell or to an organism in order to produce the therapeutic fungal lytic enzyme endogenously.

20. The method according to claim 18, wherein said fungal lytic enzyme is a β1-6 glucanase.

21. A method of treating invasive aspergillosis comprising:
(a) extracting β1-6 glucanase from Trichoderma;
(b) purifying said β1-6 glucanase to a degree of purity acceptable for therapeutic use in human subjects;
(c) formulating a pharmaceutical composition comprising said β1-6 glucanase together with a pharmaceutically acceptable carrier; and
(d) administering said pharmaceutical composition to a subject in need thereof.

22. A pharmaceutical composition comprising one or more fungal lytic enzymes administered in a therapeutically effective amount and with a degree of purity acceptable for treatment of fungal infections in human subjects.

23. The composition according to claim 22, wherein said fungal lytic enzyme is selected from the group consisting of Endo-(1-3)-β-N-glucanase, Exo-(1-3)-β-N-glucanase, Endo-(1-6)-β-N-glucanase, Exo-(1-6)-β-N-glucanase, Endo-(1-4)-β-N-glucanase, Endo-(1-4)-β-N-glucanase, Endo-(1-2)-β-N-glucanase, Endo-(1-2)-β-N-glucanase, 1-4-β-poly-N-acetyl-D-glucosaminidase, a chitinase, a chitobiosidase, a chitobiohydrolase, endo-1-4-β-poly-D-glucosaminidase, exo-1-4-β-poly-D-glucosaminidase, and a protease.

24. The composition according to claim 22, wherein the lytic enzyme is β1-6 glucanase.

25. The composition according to claim 22, wherein said lytic enzyme comprises between about 0.05% and about 99% by weight of said pharmaceutical composition.

26. The composition according to claim 22, wherein said lytic enzyme comprises between about 0.5% and about 99% by weight of said pharmaceutical composition.

27. The composition according to claim 22, wherein said therapeutically effective amount of said lytic enzyme is between about 0.1 milligram and about 2000 milligrams per day.

28. The method according to claim 22, wherein said therapeutically effective amount of said lytic enzyme is between about 1 milligram and 2000 milligrams per day.

29. The composition according to claim 22, wherein said fungus is Trichoderma.

30. The composition according to claim 22, wherein said fungus is Trichoderma harzianum.

31. The composition according to claim 22, wherein the fungal infection is caused by a species having a cell wall comprising a β-1,6-glucan.

32. The composition according to claim 22, wherein said fungal infection is caused by a species of Aspergillus.

33. The composition according to claim 22, wherein said fungal infection is caused by a species of Aspergillus fumigata.

34. The composition according to claim 22, wherein said fungal infection is invasive aspergillosis.

35. The composition according to claim 22, wherein said administering of said lytic enzyme is selected from the group consisting of (a) topical administration via a carrier material selected from a group consisting of isopropaol, glycerol, paraffin, stearyl alcohol, and polyethylene glycol; (b) parenteral; (c) intranasal; (d) inhalation; and (e) oral administration.

36. The composition according to claim 35, wherein said parenteral administration of said lytic enzyme is accomplished by subcutaneous, intramuscular and intravenous injection or by sustained release subcutaneous implant, such that a therapeutically effective amount of said lytic enzyme contacts the sites of fungal infection in vivo via systemic absorption and circulation.

37. The composition according to claim 22, wherein said lytic enzyme comprises between about 0.5% and about 20% by weight of said pharmaceutical composition for administration by injection.

38. The composition according to claim 22, wherein said lytic enzyme comprises between about 0.1% and about 50% by weight of said pharmaceutical composition for oral administration.

39. The composition according to claim 22, wherein said fungal lytic enzyme or a biologically active fragment thereof is encoded by a nucleic acid sequence contained within a recombinant plasmid.

40. The composition according to claim 22, wherein said recombinant plasmid is administered to a cell or to an organism in order to produce the therapeutic fungal lytic enzyme endogenously.

41. The method according to claim 40, wherein said fungal lytic enzyme is a β1-6 glucanase.
